(19) United States
(12) Patent Application Publication
(10) Pub. No.: US 2024/0253010 A1
(43) Pub. Date: Aug. 1, 2024

(54) ADVANCED ANTIMICROBIAL AND CHEMICAL FILTERS FOR GAS AND WATER SYSTEMS

(71) Applicant: EXPOSOME PVT. LTD., Mumbai (IN)

(72) Inventor: Prerna Goradia, Mumbai (IN)

(21) Appl. No.: 18/605,322

(22) Filed: Mar. 14, 2024

Related U.S. Application Data
(63) Continuation of application No. PCT/IN2022/050818, filed on Sep. 14, 2022.

(30) Foreign Application Priority Data
Sep. 14, 2021 (IN) 202121041437

(51) Int. Cl. B01J 20/20; A23B 7/148; A61L 9/014; B01D 53/72; B01D 53/81; B01D 53/86; B01J 20/02; B01J 20/04; B01J 20/06; B01J 20/32; B01J 21/18; B01J 23/44; B01J 37/02; C02F 1/42; C02F 101/10; C02F 101/20; G21F 9/12

(52) U.S. Cl. CPC B01J 20/20; A23B 7/148; A61L 9/014; B01D 53/72; B01D 53/81; B01D 53/864; B01J 20/0207; B01J 20/0296; B01J 20/041; B01J 20/046; B01J 20/06; B01J 20/3204; B01J 20/3223; B01J 21/18; B01J 23/44; B01J 37/0201; C02F 1/42; G21F 9/12; A61L 2209/14; A61L 2209/22; B01D 2253/102; B01D 2253/1124; B01D 2253/25; B01D 2255/1023; B01D 2257/104; B01D 2257/502; B01D 2257/7022; B01D 2257/708; B01D 2257/90; B01D 2257/91; B01D 2258/06; C02F 2101/103; C02F 2101/20

(57) ABSTRACT

Functionalized filter materials consist of carbons and ceramic substrates for special antimicrobial, phase transfer, and catalytic properties and the method of preparation thereof. The filters are coated with different types of inorganic and polymer impregnates for specific applications in air and water pollution control and catalysis. The substrates are activated by special processes and the impregnates may be reacted with certain redox systems to help increase their efficacy.

### RELATED APPLICATIONS

This application claims priority under 35 U.S.C. § 120 to, and is a continuation of, co-pending International Application PCT/IN2022/050818, filed Mar. 23, 2023 and designating the US, which claims priority to IN Application 202121041437, filed Sep. 14, 2021, such IN Application also being claimed priority to under 35 U.S.C. § 119. These IN and International applications are incorporated by reference herein in their entireties.

### FIELD

The present invention relates to the field of functionalization of filter materials. More particularly, the invention relates to functionalized, metallized and polymerized filter materials such as activated carbons and ceramic substrates including zeolites for special antimicrobial, phase transfer, and catalytic properties and the method of preparation thereof.

### BACKGROUND

Activated carbon is used to purify liquids and gases in a variety of applications, including municipal drinking water, food and beverage processing, odor removal, industrial pollution control. One gram of activated carbon may have a surface area in excess of 3,000 m2 (32,000 sq. ft). The adsorption capacity of activated carbon is largely determined by its pore structure characteristics such as its surface area, pore volume, and pore size distribution.

Further, due to its antimicrobial and antiseptic properties, antimicrobial loaded activated carbon can be used as an adsorbent for purification of domestic water. Drinking water can be obtained from natural water by treating natural water with a mixture of treated activated carbon and alumina beads for instance. This has several uses which are listed, below but not limited to: Methane and hydrogen storage; Air/water purification—absorbs volatile chemicals on molecular basis; Decaffeination; Sewage treatment; Air filters; Activated carbon is used to treat poisonings and overdoses following oral ingestion. Tablets or capsules of activated carbon are used in many countries as an over-the-counter drug to treat diarrhea, indigestion, and flatulence.

References have been made to the following literature:

Research publication by Martins A V et al in “Metal-impregnated carbon applied as adsorbent for removal of sulphur compounds using fixed-bed column technology” discusses about the efficiency of the use of activated carbon (AC) in the adsorption of sulphur compounds, especially when its surface is modified with metals. Comparing adsorption capacities of sulphur compounds from real gasoline, AC-Pd material appeared more selective than other materials, even presenting a behaviour of rapid saturation explained by the presence of other components competing for adsorption sites, reducing their effectiveness in removing sulphur compounds. Both pristine AC and Pd-AC showed good regenerability. The regenerated Pd-AC sorbent can recover about 85% of the desulphurization capacity.

US2020290014A1 relates to a method for preparing a nano-enabled activated carbon block, a nano-enabled activated carbon block produced by the method, a household water filtration system comprising the nano-enabled activated carbon block, and a method for filtering tap water using the household water filtration system are provided. The method includes contacting a solution including a metal(lic) precursor (e.g. a titanium compound and/or an iron compound and/or a zirconium compound) with activated carbon particles such that the solution fills pores of the activated carbon particles. The method further includes causing a metal (hydr)oxide (e.g. titanium dioxide and/or zirconium dioxide and/or iron oxide) to precipitate from the solution thereby causing metal oxide nanoparticles to become deposited within pores of the activated carbon particles. The method also includes preparing a nano-enabled activated carbon block from the activated carbon particles having metal oxide nanoparticles deposited within the pores thereof.

JP2002273122A relates to a method for manufacturing a baked activated carbon block filter having a large number of small pores advantageous to capture bacteria while ensuring voids, and having high capturing capacity and adsorbing capacity. The method for manufacturing the baked activated carbon block filter comprises a process for kneading compounded powdery activated carbon, which comprises a mixture of powdery activated carbon becoming a base material and ultrafine powdery activated carbon with a particle size of 20 μm or less, an inorganic binder and water, to form a granulated raw material and setting the content of the inorganic binder to 50 wt. % or more and the content of compounded powdery activated carbon to 50 wt. % or less with respect to 100 wt. % of the sum total of the inorganic powder and compounded powdery activated carbon in the raw material and a process for molding the raw material under pressure and baking the molded one to obtain the baked activated carbon block filter having a large number of pores with a fine pore size and enhanced in capturing capacity and adsorbing capacity.

It is evident that despite the widespread use of carbon block filters for water and gas filtration, there is a need for an improved filter material. In this invention, porous surfaces with high surface area such as activated carbon including graphene, ceramics, zeolites, including natural zeolites and synthetic zeolites act as absorbers and are coated with different types of organic and inorganic impregnates such as iodine, silver, copper and Al, Mn, Zn, Fe, Li, Ca, conducting polymers and used for specific application in air and water pollution control and catalysis. Specific materials such as strong oxidizing agents and reagents are impregnated and coated on absorbers such as these zeolites and alumina beads.

The information disclosed in this background of the disclosure section is only for enhancement of understanding of the general background of the invention and should not be taken as an acknowledgement or any form of suggestion that this information forms the prior art already known to a person skilled in the art.

### SUMMARY

The principal object of the present invention to provide a composition and method of preparing metallized and polymerized filter materials with special antimicrobial, gas-phase transfer, and catalytic properties.

Another object of the present invention is to provide a composition for higher absorption of organic contaminants that can be used for improving the food storage and such applications by absorbing gases that cause spoilage.

The present invention attempts to overcome the problems faced in the prior art, and discloses a composition and method of preparing metallized and polymerized filter materials with special antimicrobial, gas-phase transfer, and catalytic properties.

In accordance with the embodiments of the present invention, the invention relates to a process for making active molecular filters, comprising the steps of taking a measured amount of a substrate in a reaction vessel and activating it, followed by functionalization of the substrate by mixing with measured amount of at least one impregnate to form a molecular filter media. Further, a reducing or oxidizing agent is added at the functionalization step as per the required application and activity of the molecular filters and a binder may also be added to immobilize the soluble ions inside the molecular filter media.

In accordance with the embodiments of the present invention, the invention discloses a process for making active molecular filters, where the substrate comprises porous surfaces with high surface areas selected from a group comprising carbons including graphene, ceramics, zeolites both natural and synthetic zeolite, silica molecular sieves, microporous phosphate oxides and even organic-inorganic hybrid materials such as metal organic frameworks (MOF) and combinations thereof.

In accordance with the embodiments of the present invention, the substrate is activated by heating in a furnace up to 1200° C. till calcination but without melting to increase the surface area of the substrate.

In accordance with the embodiments of the present invention, the invention discloses a process where the impregnate is selected from a group comprising elements or salts of silver, zinc, copper, aluminium, manganese, iron, calcium, palladium, iodine and polymers both conducting and water-soluble polymers such as polyvinyl pyrrolidone, poly vinyl alcohol, polyaniline, poly pyrrole and combinations thereof.

In accordance with the embodiments of the present invention, the reducing agent is selected from a group comprising formaldehyde, hypophosphite, ascorbic acid, borates, metabisulphites and combinations thereof.

In accordance with the embodiments of the present invention, in the reaction vessel which is often a rotating type mixer, the impregnate and the substrates are mixed together along with solvents to form a “coating” and or impregnate on the substrate. The key is to enable gentle infusion into the substrate materials without mechanically damaging them and this is enabled by the tumbling action of the mixer.

In accordance with the embodiments of the present invention, the invention discloses the process, where functionalization of the substrate is done by any of the techniques such as electrolysis, electroless deposition by oxidation-reduction reaction, extrusion techniques and combinations, but not limited to, depending upon the surface of the substrate and the activity required.

In accordance with the embodiments of the present invention, the invention discloses the process, in which the impregnates are “reduced” or “oxidized” on the surface of the porous materials using reducing agents and oxidizing agents for making a metal or polymer system in the molecular filters. In another embodiment, instead of reagents, electrons may also be used in the present invention.

In accordance with the embodiments of the present invention, in the extrusion process, the materials are “extruded” into various shapes and forms to improve their surface area and their interactions with the reagents for cleaning the air and water systems of pollutants. The functionalized processes in turn lead to extremely porous materials with applications in abatement and catalytic conversion of pollutants. The actives may be directly extruded with minimal substrates and binders, to maximize the stoichiometric reactions of the pollutants. Such as, for the reaction of sodium metabolites with the oxygen the extrusion material can have a very large and major proportion of the reagent itself.

In accordance with the embodiments of the present invention, the invention provides a composition for active molecular filtering material comprising of at least a porous substrate material and at least an impregnate. Further, the impregnate is a metal, organic or inorganic impregnate for functionalization of the porous substrate.

In accordance with the embodiments of the present invention, the molecular filter is in the form of a filter block, loose media, or a coating on a fabric, or filled inside a perforated casing, or combinations thereof.

In accordance with the embodiments of the present invention, the “blocks” of the molecular filters are made using molding techniques with the use of binders and resins, followed by baking to burn off the binders leaving a high surface area porous filter block. In another embodiment, the melt flow rate of binder is 2-5 g/10 minutes and the mixture of molecular filter media and the binders is heated to a temperature in the range of 150-950° C. for the desired shape and porosity of the molecular filter blocks.

The foregoing summary is illustrative only and is not intended to be in any way limiting. In addition to the illustrative aspects, embodiments, and features described above, further aspects, embodiments, and features will become apparent by reference to the drawings and the following detailed description.

### DETAILED DESCRIPTION

While the embodiments of the disclosure are subject to various modifications and alternative forms, specific embodiments thereof have been shown by way of example in the figures and will be described below. It should be understood, however, that it is not intended to limit the disclosure to the particular forms disclosed, but on the contrary, the disclosure is intended to cover all modifications, equivalents, and alternatives falling within the scope of the disclosure. Further, the phraseology and terminology employed in the description is for the purpose of description only and not for the purpose of limitation.

The terms “comprises”, “comprising”, or any other variations thereof used in the disclosure, are intended to cover a non-exclusive inclusion, such that a device, apparatus, system, assembly, method that comprises a list of components or a series of steps that does not include only those components or steps but may include other components or steps not expressly listed or inherent to such apparatus, or assembly, or device. In other words, one or more elements or steps in a system or device or process proceeded by “comprises . . . a” or “comprising . . . of” does not, without more constraints, preclude the existence of other elements or additional elements or additional steps in the system or device or process as the case may be.

The primary object of the present invention is to provide a composition and method of preparing metallized and polymerized filter materials with special antimicrobial, gas-phase transfer, and catalytic properties.

In accordance with the embodiments of the present invention, the invention relates to a process for making active molecular filters, comprising the steps: (a) taking a measured amount of a substrate in a reaction vessel and activating it; (b) functionalization of the substrate by mixing with measured amount of at least one impregnate to form a molecular filter media; wherein a reducing or oxidizing agent is added at the functionalization step as per the required application and activity of the molecular filters and a binder may also be added to immobilize the soluble ions inside the molecular filter media.

In an embodiment of the present invention, the invention discloses a process for making active molecular filters, where the substrate comprises porous surfaces with high surface areas selected from a group comprising carbons including graphene, ceramics, zeolites both natural and synthetic zeolite, silica molecular sieves, microporous phosphate oxides and even organic-inorganic hybrid materials such as metal organic frameworks (MOF) and combinations thereof.

In another embodiment of the present invention, the substrate is activated by heating in a furnace up to 1200° C. till calcination but without melting to increase the surface area of the substrate.

In another embodiment of the present invention, the invention discloses a process where the impregnate is selected from a group comprising elements or salts of silver, zinc, copper, aluminium, manganese, iron, calcium, palladium, iodine and polymers both conducting and water-soluble polymers such as polyvinyl pyrrolidone, poly vinyl alcohol, polyaniline, poly pyrrole and combinations thereof.

In still another embodiment of the present invention, the reducing agent is selected from a group comprising formaldehyde, hypophosphite, ascorbic acid, borates, metabisulphites and combinations thereof.

In another preferred embodiment of the present invention, the invention discloses a process where in the reaction vessel which is often a rotating type mixer, the impregnate and the substrates are mixed together along with solvents to form a “coating” and or impregnate on the substrate. The key is to enable gentle infusion into the substrate materials without mechanically damaging them due to the tumbling action of the mixer.

In an embodiment of the present invention, the invention discloses the process, where functionalization of the substrate is done by any of the techniques such as electrolysis, electroless deposition by oxidation-reduction reaction, extrusion techniques and combinations, but not limited to, depending upon the surface of the substrate and the activity required.

In yet another embodiment of the present invention, the invention discloses the process, in which the impregnates are “reduced” or “oxidized” on the surface of the porous materials using reducing agents and oxidizing agents for making a metal or polymer system in the molecular filters. In another embodiment, instead of reagents, electrons may also be used in the present invention.

In a preferred embodiment of the present invention, in the extrusion process, the materials are “extruded” into various shapes and forms to improve their surface area and their interactions with the reagents for cleaning the air and water systems of pollutants. The functionalized processes in turn lead to extremely porous materials with applications in abatement and catalytic conversion of pollutants. The actives may be directly extruded with minimal substrates and binders, to maximize the stoichiometric reactions of the pollutants. Such as, for the reaction of sodium metabolites with the oxygen the extrusion material can have a very large and major proportion of the reagent itself.

In an exemplary embodiment of the present invention, the invention provides a composition for active molecular filtering material comprising of at least a porous substrate material; and at least an impregnate, where the impregnate is a metal, organic or inorganic impregnate for functionalization of the porous substrate.

In yet another embodiment of the present invention, the molecular filter is in the form of a filter block, loose media, or a coating on a fabric, or filled inside a perforated casing, or combinations thereof.

In still another embodiment of the present invention, the “blocks” of the molecular filters are made using molding techniques with the use of binders and resins, followed by baking to burn off the binders leaving a high surface area porous filter block. In another embodiment, the melt flow rate of binder is 2-5 g/10 minutes and the mixture of molecular filter media and the binders is heated to a temperature in the range of 150-950° C. for the desired shape and porosity of the molecular filter blocks.

According to this invention, there is provided a method of preparing metallized and polymerized filter materials with special antimicrobial, gas-phase transfer, and catalytic properties.

Substrate for the material comprises of porous surfaces such as activated carbon including graphene, zeolites, ceramics have sigh surface area. Porous materials contain voids (or pores), either in isolation or interconnected to form complex networks of channels, which are filled with fluid under normal atmospheric conditions, e.g. air, liquid water. The inorganic porous materials include natural zeolites, synthetic zeolite (from low-siliceous zeolite to high-siliceous zeolite), pure silica molecular sieve, microporous phosphate oxides and even organic-inorganic hybrid materials such as metal organic frameworks (MOF), finely grinded metal powder mixed with carbon as a carbon block and are activated by heating. Heating the material further increases the surface area, in turn making more area available for reacting with the pollutants.

Inorganic impregnates such as iodine, silver, copper and Al, Mn, Zn, Fe, Li, Ca, Pd and conducting polymers are deposited on this activated material as metals, for functionalization of the carbon/zeolites/ceramics and works by adsorbing and reacting with the pollutants. Water soluble polymers can also be chosen from polyvinyl pyrrolidone, polyvinyl alcohol and the like. Further, if the material is soluble then it can be made insoluble for a stable and long-term sustenance activity. Selection of impregnates and polymers is dependent upon the application required. Because of metallization, the analytes get trapped/adsorbed on the surface area, metal on the surface immobilizes the analytes and converts it into the inert or less toxic specie. In turn results in diminished activation energy.

The invention discloses a process, for making metal-impregnated block filter, comprising the steps of contacting the substrate let us say the ceramic powders with salts or elements of silver, zinc, and/or copper optionally in the presence of reducing agents, to form an aqueous mix, followed by mixing the said mix with a binder, having melt flow rate between 2-5 g/10 min to form a bonded mixture. Further, the bonded mixture is added to a mold and the mold with the bonded mixture is heated to a temperature in the range of 150 to 950 degree C. in order to obtain a block filter in the mold. The filter should not go up till the melting step or it would lose the porosity. Lastly, de-molding the carbon block filter formed from the mold.

Metallization/functionalization is done by techniques such as ball mixer or pan mixer, tumble dry, extrusion techniques and combinations, but not limited to. In the mixer, the reactants and the substrate may be mixed together in a mixer along with solvents such as water to form a “coating” on the substrate. The reagents will also percolate and occupy the many “sites” on the porous materials. The drying temperature is generally between 100 to 900° C. where the reagent is properly “fixed” onto the substrate.

The materials could also be “extruded” in the functionalization process simply termed here as the extrusion process. Porous materials with applications in catalysis, filtration can be processed by extrusion of ceramic emulsions, obtained by emulsification of ceramic powder suspensions, after sintering at different temperatures. The emulsification of the ceramic suspension in paraffin with a melting point higher than room temperature is the key for the success of this method due to the freezing of the organic mixture allowing stability of matrix during the extrusion step. The reagents could be mixed in with the ceramic materials and in certain cases the reagents themselves could be extruded also.

The ceramic filter blocks can also be prepared by using the functionalized materials where a binder can be mixed into them. The binder could be from monomers having ester or amide functional groups, poly(vinyl amine), poly(vinyl formamide) or a copolymer of vinyl alcohol and vinyl amine for instance.

The already prepared carbon blocks were dunked in metal salts such as copper sulphate and a current was applied to the carbon block that served as the cathode. In some cases, a copper block was used as an anode. Deposition of copper metal took place inside and on the carbon block. In the next example instead of electrons a reducing agent such as “hypophosphite” and formaldehyde were used along with the copper solution. In this fashion the copper metal was reduced onto the carbon blocks. These types of carbon had very prominent antimicrobial properties as described in the first two examples below.

### EXAMPLES

Example 1: Microbiological studies was done for the ‘activated carbon block’ developed according to a process of this invention. For this, time kill study of a sample of E. coli, using ASTM E2315—Suspension Time-Kill Test, was observed. The sample showed microbial activity. For preparation of culture, in this study, a pure culture of E. coli was streaked on Soybean Casein Digest Agar plates and incubated at 37° C. for up to 2 days. Following incubation, the surface of agar plate was scraped. The growth suspension was adjusted to a concentration of 106 cfu/ml. Test and control substances were dispensed in identical volumes to sterile test tubes. Independently, ‘Test substances’ and ‘Control substances’ were inoculated with the test microorganism, mixed, and incubated. Control suspensions were immediately plated to represent the concentration present at the start of the test, or time zero. At the conclusion of each contact time, a volume of the liquid test solution was neutralized. Dilutions of the neutralized test solution were placed on to appropriate agar plates and incubation temperatures to determine the surviving microorganisms at the respective contact times. Reductions of microorganisms were calculated by comparing initial microbial concentrations to surviving microbial concentrations. All tests were performed in duplicates and counts averaged (Table 1).

TABLE 1
Results for the activated carbon block

| Test | Test | Contact | | % | Log₁₀ |
|---|---|---|---|---|---|
| Organism | Material | Time | CFU/ml | Reduction | Reduction |
| Escherichia | Control | Time | 4.2 × 10⁶ | not | not |
| coli | | zero | | applicable | applicable |
| | Activated | 30 Min | 1 | 99.9999% | 6.623 |
| | Carbon | | | | |
| | Control | | | | |

It was inferred that the test sample-impregnated activated Carbon, according to the first process, of this invention, shows antimicrobial activity against Escherichia coli when exposed for 30 mins.

Example 2: According to an exemplary embodiment, Copper was impregnated on the carbon block and when the reaction took place, the copper blue electrolyte solution turned colorless. After washing and putting a drop of acid, the solution, again, turned blue, indicating the presence of copper as the blue colored copper salt and confirming the functionalization of the matrices with the metals. X-ray spectroscopy results showed the presence of copper. Microbiological results, with 30-minute contact time of E. coli was observed to be positive with 6 log reduction.

Example 3: According to an embodiment of the present invention, it was observed that the bound carbon block filters with relatively low level of variation in metal content across the blocks, relatively lower deviation from the theoretical metal content, and relatively lower leach-rate of metal from the blocks during use, could be obtained by a process in which the activated carbon blocks were used as cathodes in electrolysis reactions where the controlled metallization of the blocks could occur. A formulation developed according to this process of this invention, time kill study of a sample of E. coli, using ASTM E2315—Suspension Time-Kill Test, was conducted. The test sample was named ‘Activated Carbon control with Antimicrobial Agent (Treated)’ and the reference sample was names ‘Activated Carbon control without Antimicrobial Agent (Untreated)’. Sample quantity, used, was 1 piece each (Table 2). Culture Preparation and the experiment was performed as per the previous example. Reductions of microorganisms were calculated by comparing initial microbial concentrations to surviving microbial concentrations. All tests were performed in duplicates and counts averaged. The microbiological result, after this process, showed antimicrobial activity. Activated carbon, that was “impregnated with copper” using the process, showed 99.9999 reduction.

TABLE 2
Results for the activated carbon blocks with the electrolysis method

| Test | Test | Contact | | Log | Log | % |
|---|---|---|---|---|---|---|
| Organism | Material | Time | CFU/ml | Value | Reduction | Reduction |
| Escherichia | Untreated | Time zero | 3700000 | 6.568 | — | — |
| coli | Control | | | | | |
| | Treated | 15 Min | <1 | 0 | 6.568 | 99.99997 |
| | Sample | | | | | |

It can be inferred that the test sample ‘Activated Carbon control with Antimicrobial Agent (Treated)’ when compared with the test sample ‘Activated Carbon control without Antimicrobial Agent (Untreated)’ showed antimicrobial activity against Escherichia coli when exposed up to 15 mins.

Example 4: A lab-scale filtering system was set up to study VOC and formaldehyde removal efficiency of the metallized activated carbon in combination with alumina impregnated with oxidizers such as permanganate and ceric ammonium nitrate. The efficiency of these two types of filtering media was studied and there was a complete removal of the VOC and formaldehyde from the air. It was observed that the process, of this invention, offered high-cost savings due to reduction in energy consumption. The filter materials, according to this invention, helped in removing harmful and unpleasant particles, gases, odours, bacteria, and viruses from the environment. The applications could be several including corrosion prevention of devices and machines, reduced odors, increased equipment reliability, preservation of artifacts such as in museums, increased shelf-life of food items and daily consumables, and more.

Example 5: Metallization of activated carbon materials with catalysts such as palladium and similar group materials for the removal of carbon monoxide this is an example of catalyzed reactions where the catalysis property of the metal center, along with the high adsorption area of the activated carbon helps in the reaction. There is a need to develop catalysts that are not poisoned by CO and become active at low temperatures. The palladium in the process was made into a colloidal solution using palladium chloride plus stannous chloride and special complexing agents such as tartaric acid. The stannous chloride reduced the palladium on the activated carbon surface and the palladium centers in turn helped to oxidize the carbon monoxide. Atomically dispersed Pd, bound to surface oxygen atoms on carbon, resisted the CO poisoning and was able to achieve high activity for CO oxidation at low temperatures. This type of filter has several applications in defense and submarines etc.

Example 6: Removal of radioactivity. A special matrix was prepared by mixing activated carbon with potassium hydroxide and iodide. Another layer was the 4% magnesium chloride with diluted sodium hydroxide. The described matrix was then mixed with a water-soluble polymer such as polyvinyl pyrrolidine to securely bind the reagents and high surface substrates to withstand the media where the dissolution is happening. The two were introduced as layers in the filters and helped in lowering the radioactive uranium in the samples (Table 3). The results are for the solutions of the water spiked with uranium prior to passing through the column and after passing through the column. The water was flowed through the column at 5 ml/min. The iodide ion is a strong reducing agent; that is, it readily gives up one electron and therefore under the optimum environments can bind with the uranium easily immobilizing it into the matrix.

TABLE 3
Results for the radioactivity removal experiment

| | | | | Results of |
|---|---|---|---|---|
| S. No | Chemical parameters | Units | Methods | Analysis |
| 1 | Uranium (Spiked) | ppb | ICP-MS | 148 |
| 2 | Uranium (after passing through | ppb | ICP-MS | <10 |
| | the cartridge) | | | |

Example 7: Removal of heavy metals. Fine alumina powder was mixed with manganese and other transition metal salts and heated in a furnace up to 1200° C. This mixture was excellent for the removal of lead and other heavy metals from the drinking waters. The mixture was mixed with the carbon and diminished amount of lead was recorded after the lead was allowed to pass through the above media (Table 4). There is an exchange reaction of the heavy metals with the active metal ions in the high surface area media that gets formed. Several toxic materials such as lead, cadmium, arsenic, mercury and others can be removed using this filter matrix.

TABLE 4
Data for the radioactivity removal application of the filter matrix

| | |
|---|---|
| As it is Pb(II)(lead) in the water (Before | 207 ppm |
| flowing through the media) | |
| The amount of lead after passing | 96 pm |
| through above media | |

Example 8: Compositions as ethylene absorbers: Fruits are either ethylene producers or absorbers. Apples, bananas, melons, pears and peaches are ethylene producers. For prolonging the life of such fruits, the ceramic beads were prepared that had a high content of oxidizing agents such as permanganates and ceric ammonium nitrates and combinations ranging from 6 to 20% and it was observed that about 6% KMno4 in the alumina beads helped to prolong the life of bananas for instance.

Example 9: Compositions to control oxygen and humidity: Another gas to control is oxygen. Special ceramic reagent beads were prepared by using 10 to 20% sodium metabisulphite with alumina balls. These “reducers” helped the oxygen content and prolonged the life of certain fruits especially tomatoes. Humidity control was also achieved using very high surface area molecular beads (special ceramics) that were “baked” in the furnace at high temperatures up to 900° C. This media helped to absorb and desorb the humidity prolonging the life of fruits.

Polymer coated carbon by electrolysis using conducting polymers in which porous carbons or absorbent matrices can be coated with a biocompatible polymer to form a smooth, permeable layer without clogging the pores. The process involves the electrolytic deposition of the polymer on the substrates which can be used for hemoperfusion. Hemoperfusion is a treatment method in which blood is drawn from the patient's body and externally passed through an adsorbent to remove toxic substances. This kind of simulated matrix has also been prepared and demonstrated removal of toxins with molecular weights ranging up to several thousand daltons that bind to the coated substrates. Binding is through physical adsorption and may depend on molecular weight and lipophilicity. Generally lower molecular-weight toxins are adsorbed.

In warfare where multiple people are sitting in a tank also this kind of materials can have a very big impact as it can remove the toxic warfare agents in closed environments. The applications of the metal impregnated carbons on gas filters are noteworthy. An ABEK filter for instance absorbs dangerous gases and vapours, enabling the user to breathe safely. Besides, since metals impregnated into the filters, there are no concerns of leaching out into the solution/water. Catalysts such as zinc sulphate, copper sulphate and molybdenum oxide were used to prepare the carbons that blocked the incoming gases and vapors by chemisorption.

It will be further appreciated that functions or structures of a plurality of components or steps may be combined into a single component or step, or the functions or structures of one-step or component may be split among plural steps or components. The present invention contemplates all of these combinations. Unless stated otherwise, dimensions and geometries of the various structures depicted herein are not intended to be restrictive of the invention, and other dimensions or geometries are possible. In addition, while a feature of the present invention may have been described in the context of only one of the illustrated embodiments, such feature may be combined with one or more other features of other embodiments, for any given application. It will also be appreciated from the above that the fabrication of the unique structures herein and the operation thereof also constitute methods in accordance with the present invention. The present invention also encompasses intermediate and end products resulting from the practice of the methods herein. The use of “comprising” or “including” also contemplates embodiments that “consist essentially of” or “consist of” the recited feature.

Although embodiments for the present invention have been described in language specific to structural features, it is to be understood that the present invention is not necessarily limited to the specific features described. Rather, the specific features and methods are disclosed as embodiments for the present invention. Numerous modifications and adaptations of the system/component of the present invention will be apparent to those skilled in the art, and thus it is intended by the appended claims to cover all such modifications and adaptations which fall within the scope of the present invention.

Advantages: Porous material with high surface area, comprise of a large number of reacting sites with high content of reagents, so that maximum amount of pollutants can be trapped; Carbon monoxide is abated; Manganate and ceric ammonium nitrate coated ceramic beads to remove ethylene; Sodium metabisulfite impregnated beads to remove oxygen; Humidity control; Removal of radioactivity; Removal of heavy metals; and Metals impregnation, in turn with no leaching out of the metals and stable coating.

1. A method of making an active molecular filter, the method comprising:
activating a substrate in a reaction vessel; and
functionalizing the substrate by mixing in an impregnate to form a molecular filter media, wherein the functionalizing includes further adding a reducing or oxidizing agent based on an activity of the molecular filter to be achieved.

2. The method of claim 1, wherein the substrate includes at least one of, a zeolite, a silica molecular sieve, phosphate oxide, and an organic-inorganic hybrid material, all having porous surfaces and high surface areas.

3. The method of claim 2, wherein the substrate includes at least one of, graphene, ceramic, natural zeolite, synthetic zeolite, microporous phosphate oxide, and a metal organic framework.

4. The method of claim 1, wherein the activating includes heating the substrate in a furnace up to 1200° C. to calcinate and not melt the substrate, wherein the heating increases a surface area of the substrate.

5. The method of claim 1, wherein the impregnate is at least one of, elemental silver, a salt of silver, elemental zinc, a salt of zinc, elemental copper, a salt of copper, elemental aluminum, a salt of aluminum, elemental manganese, a salt of manganese, elemental iron, a salt of iron, elemental calcium, a salt of calcium, elemental palladium, a salt of palladium, elemental iodine, a salt of iodine, polyvinyl pyrrolidone, polyvinyl alcohol, polyaniline, and polypyrrole.

6. The method of claim 1, wherein the reducing or oxidizing agent is at least one of, formaldehyde, hypophosphite, ascorbic acid, a borate, and metabisulphite.

7. The method of claim 1, wherein the mixing in the impregnate includes mixing in a solvent to coat and/or impregnate the substrate without mechanically reshaping the substrate or the impregnate.

8. The method of claim 1, wherein the functionalizing includes at least one of, electrolysis, electroless deposition by an oxidation-reduction reaction, and extrusion.

9. The method of claim 8, wherein the functionalization reduces or oxidizes the impregnate on a porous surface of the substrate.

10. The method of claim 8, wherein in the functionalizing includes extrusion of the substrate to increase a surface area of the substrate and interaction rate of the active molecular filter.

11. The method of claim 8, wherein the functionalizing includes electrolysis, wherein the substrate is carbon used as an anode or cathode in the electrolysis, and wherein the impregnate is copper impregnated in the carbon from a copper solution by the electrolysis, and wherein the reducing or oxidizing agent is hypophosphite and/or formaldehyde.

12. The process of claim 1, further comprising:
adding a binder to the molecular filter media to immobilize soluble ions inside the molecular filter media.

13. The filter of claim 12, wherein the binder has a melt flow rate of about 0.2-0.5 g/minute, and wherein the method further comprises:
heating the molecular filter media and the binder to about 150-950° C. to achieve a desired shape and porosity.

14. An active molecular filter comprising:
a porous substrate material; and
an impregnate, wherein the impregnate is at least one of, a metal, an organic molecule, and an inorganic molecule, and wherein the impregnate functionalizes the porous substrate.

15. The filter of claim 14, wherein the molecular filter is at least one of, a filter block, loose media, a coating on a fabric, and a perforated casing filler.

16. The filter of claim 15, wherein molecular filter is a filter block made using molding and baking, wherein a surface of the filter block is free of resins.
